(19) United States
(12) Patent Application Publication
(10) Pub. No.: US 2020/0321128 A1
(43) Pub. Date: Oct. 8, 2020

(54) DATA PROCESSING DEVICE, DATA PROCESSING METHOD AND NON-TRANSITORY STORAGE MEDIUM STORING DATA PROCESSING PROGRAM

(71) Applicant: OMRON HEALTHCARE Co., Ltd., Kyoto (JP)

(72) Inventors: Hiroshi NAKAJIMA, Kyoto-shi (JP); Hirotaka WADA, Kyoto-shi (JP); Daisuke NOZAKI, Kyoto (JP); Tamio UEDA, Kyoto-shi (JP)

(21) Appl. No.: 16/906,420

(22) Filed: Jun. 19, 2020

Related U.S. Application Data
(63) Continuation of application No. PCT/JP2018/046250, filed on Dec. 17, 2018.

(30) Foreign Application Priority Data
Dec. 27, 2017 (JP) 2017-252600

(51) Int. Cl. G16H 50/20; A61B 5/00; A61B 5/021

(52) U.S. Cl. CPC G16H 50/20; A61B 5/7246; A61B 5/0022; A61B 5/02108; A61B 5/7275

(57) ABSTRACT

A data processing device includes an acquisition unit configured to acquire first biometric data related to a first subject, a retrieval unit configured to retrieve second biometric data that is related to a second subject and has a degree of similarity to temporal change of the first biometric data that satisfies a first condition, and a prediction unit configured to predict onset of a particular disease in the first subject based on information indicating onset of the particular disease in the second subject associated with the second biometric data.

### CROSS-REFERENCE TO RELATED APPLICATIONS

This is a continuation of International Application 2017-252600, with an international filing date of Dec. 27, 2017, and International Application PCT/JP2018/046250, with an international filing date of Dec. 17, 2018, filed by applicant, the disclosure of which is hereby incorporated by reference in its entirety.

### TECHNICAL FIELD

The present invention relates to a technology for predicting the onset of a disease in a subject from biometric data.

### BACKGROUND ART

As disclosed in JP 2016-64125 A, systems are being developed to predict the risk of onset for cerebrovascular diseases from fluctuation in blood pressure and fluctuation in pulse wave waveforms based on brainwave information that is regularly continuously acquired from a subject. The system disclosed in JP 2016-64125 A predicts the risk of onset by determining the amount of blood pressure fluctuation and the amount of fluctuation in pulse wave waveforms.

Entities such as medical facilities and insurance companies have investigated the use of technology that predicts the onset of a disease to prevent the onset of that disease. In medical facilities, such technology is expected to suppress an increase in the number of patients who develop diseases. In insurance companies, the technology is expected to reduce payment of insurance claims associated with the onset of diseases.

### SUMMARY OF INVENTION

However, the likelihood of developing a cerebrovascular disease varies depending on how long it takes until the amount of blood pressure fluctuation and the amount of pulse wave waveform fluctuation exceed a normal range. Because the likelihood of developing a cerebrovascular disease varies, accuracy when predicting the risk of onset for cerebrovascular diseases also varies. Thus, the amount of blood pressure fluctuation and the amount of pulse wave waveform fluctuation disclosed in JP 2016-64125 A are not sufficient values for predicting the risk of onset for cerebrovascular diseases.

An aim of the present invention is to provide a data processing device, a data processing method, and a non-transitory storage medium storing a data processing program that can more accurately predict the onset of disease in a subject from biometric data.

A first aspect of the present disclosure is a data processing device including an acquisition unit configured to acquire first biometric data related to a first subject, a retrieval unit configured to retrieve, from among one or more pieces of biometric data, second biometric data that is related to a second subject and has a degree of similarity to temporal change of the first biometric data that satisfies a first condition, the one or more pieces of biometric data related to one or more subjects different from the first subject and associated with information indicating onset of a disease in each of the one or more subjects, and a prediction unit configured to predict onset of a particular disease in the first subject based on information indicating onset of the particular disease in the second subject associated with the second biometric data.

According to the first aspect, the data processing device can more accurately predict the onset of a particular disease in the first subject by retrieving the second biometric data for which a degree of similarity to temporal change of the first biometric data satisfies a first condition.

A second aspect of the present disclosure is the above-described first aspect, in which the retrieval unit is configured to retrieve the second biometric data that is related to the second subject and has a degree of similarity to an attribute of the first subject that satisfies a second condition.

According to the second aspect, the data processing device can more accurately predict the onset of a particular disease in the first subject by retrieving the second biometric data in consideration of attributes of the first subject. This is because, even when there are a plurality of pieces of biometric data indicating the onset of a particular disease, the temporal change of each piece of biometric data varies depending on the attributes of the subject related to each piece of biometric data.

A third aspect of the present disclosure is the first aspect, further including a creation unit configured to create a first message including advice for the first subject based on a prediction result that predicts onset of the particular disease in the first subject, and an output unit configured to output the first message.

According to the third aspect, the data processing device can provide the first subject with the first message before the first subject develops a particular disease. As a result, the first subject can prevent the onset of a particular disease by referring to the advice included in the first message.

A fourth aspect of the present disclosure is the above-described third aspect, in which the creation unit is configured to create the first message including information based on lifestyle of the second subject as the advice.

According to the fourth aspect, the first subject can refer to the information based on the lifestyle of the second subject who has developed a particular disease to reconsider their lifestyle and prevent the onset of the particular disease.

A fifth aspect of the present disclosure is the above-described third aspect, in which the output unit is configured to output the first message based on an instruction indicating permission of medical personnel to output the first message.

According to the fifth aspect, the data processing device can present the first message to the first subject in compliance with laws and regulations even when permission of medical personnel is required by laws and regulations to present the first message to the first subject. As a result, the first subject can obtain a high quality first message based on the permission of medical personnel.

A sixth aspect of the present disclosure is the above-described third aspect, further including a determination unit configured to determine whether the first biometric data after the first message is output is similar to either the second biometric data or third biometric data that is related to a third subject and not associated with information indicating the onset of a disease, in which the creation unit is configured to create a second message including advice indicating different content for the first subject according to a determination result indicating whether temporal change of the first biometric data after the first message is output is similar to either the second biometric data or the third biometric data, and the output unit is configured to output the second message.

According to the sixth aspect, the data processing device can determine whether the first subject is still likely to develop a particular disease after the first message is output. As a result, the first subject can refer to the second message output after the first message to understand whether they are still likely to develop a particular disease.

A seventh aspect of the present disclosure is a data processing method including acquiring first biometric data related to a first subject, retrieving, from among one or more pieces of biometric data, second biometric data that is related to a second subject and has a degree of similarity to temporal change of the first biometric data that satisfies a first condition, the one or more pieces of biometric data related to one or more subjects different from the first subject and associated with information indicating onset of a disease in each of the one or more subjects, and predicting onset of a particular disease in the first subject based on information indicating onset of the particular disease in the second subject associated with the second biometric data.

According to the seventh aspect, the data processing method can achieve the same effects as the first aspect described above.

An eighth aspect of the present disclosure is a non-transitory storage medium storing a data processing program causing a computer to function as each unit included in the data processing device of any one of the first to sixth aspects.

According to the eighth aspect, the non-transitory storage medium storing a data processing program can achieve the same effects as the first aspect described above.

According to the present invention, a technology that can more accurately predict the onset of a disease in a subject from biometric data can be provided.

### BRIEF DESCRIPTION OF DRAWINGS

FIG. 1 is a diagram schematically illustrating an application example of a server according to an embodiment.

FIG. 2 is a diagram illustrating an example of a data transmission system including the server according to the embodiment.

FIG. 3 is a block diagram illustrating an example of the hardware configuration of the server according to the embodiment.

FIG. 4 is a diagram illustrating an example of the configuration of a first management table according to the embodiment.

FIG. 5 is a block diagram illustrating an example of the software configuration of the server according to the embodiment.

FIG. 6 is a graph showing an example of comparison between first blood pressure data and reference data according to the embodiment.

FIG. 7 is a flowchart illustrating an example of a prediction operation for onset of a disease according to the embodiment.

FIG. 8 is a graph showing an example of output timing of a first message according to the embodiment.

FIG. 9 is a diagram illustrating an example of a second management table according to a modified example of the embodiment.

FIG. 10 is a block diagram illustrating an example of the software configuration of a server according to a modified example of the embodiment.

FIG. 11 is a graph showing an example of comparison between first blood pressure data and reference data according to a modified example of the embodiment.

FIG. 12 is a flowchart illustrating an example of a prediction operation for onset of a disease according to a modified example of the embodiment.

### DESCRIPTION OF EMBODIMENTS

An embodiment according to the present disclosure (hereinafter, also referred to as “the present embodiment”) will be described below with reference to the drawings. The present embodiment described below is merely illustrative in all respects. Note that elements that are the same as or similar to elements that have already been described will be given the same or similar reference numerals, and duplicate descriptions will generally be omitted. Further, data appearing in the present embodiment is described using natural language. More specifically, the data is specified by a pseudo language, commands, parameters, or a machine language.

### § 1 APPLICATION EXAMPLE

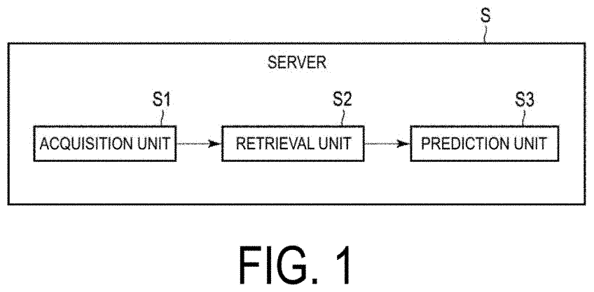

FIG. 1 is a block diagram illustrating an application example of a server S according to the present embodiment.

The server S includes an acquisition unit S1, a retrieval unit S2, and a prediction unit S3.

The acquisition unit Si acquires first blood pressure data related to a first subject.

The retrieval unit S2 retrieves second blood pressure data related to a second subject different from the first subject. The second blood pressure data has a degree of similarity to temporal change of the first blood pressure data that satisfies a first condition.

The prediction unit S3 predicts the onset of a particular disease in the first subject based on information indicating the onset of the particular disease in the second subject associated with the second blood pressure data.

Thus, the server S can more accurately predict the onset of a disease in the first subject.

### § 2 CONFIGURATION EXAMPLE

### Data Transmission System

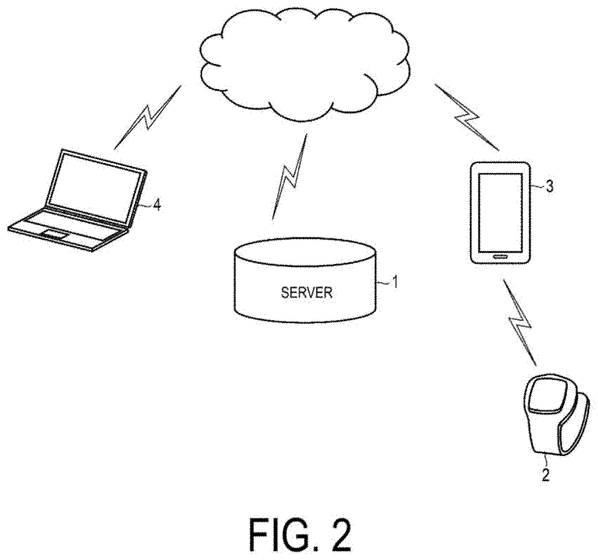

FIG. 2 is a block diagram illustrating an example of a data transmission system including a server 1 according to the present embodiment.

The data transmission system includes the server 1, a blood pressure monitor 2, a mobile terminal 3, and an information processing terminal 4.

The server 1 predicts the onset of a disease in a target person. The server 1 stores target data. The target data is blood pressure data related to the target person. Hereinafter, the target person is also referred to as a first subject. The target data is also referred to as first blood pressure data.

The server 1 stores one or more pieces of first reference data. The one or more pieces of first reference data are blood pressure data related to one or more subjects different from the first subject. Each of the one or more pieces of first reference data is associated with information indicating the onset of a disease in each subject. The information indicating the onset of a disease is based on diagnostic results of a physician.

The server 1 is used in, for example, a medical facility or an insurance company. The server 1 is an example of a data processing device. The configuration of the server 1 will be described later.

The blood pressure monitor 2 is a blood pressure monitor capable of continuously measuring beat-to-beat blood pressure of the first subject. The blood pressure monitor 2 is, for example, a wearable blood pressure monitor. The blood pressure monitor 2 acquires blood pressure data by measuring the blood pressure of the first subject. The blood pressure data is an example of biometric data. The blood pressure monitor 2 transmits the blood pressure data of the first subject to the mobile terminal 3 by using near-field wireless communication. Near-field wireless communication is Bluetooth (trade name) communication, but is not limited thereto.

The blood pressure data can include, but is not limited to, values of systolic blood pressure SBP and diastolic blood pressure DBP, and pulse rate. Note that the term “blood pressure value” used in the present embodiment refers to both the value of systolic blood pressure SBP and the value of diastolic blood pressure DBP. The blood pressure data also includes the date and time of blood pressure measurement. The date and time of measurement is detected by a clock function implemented in the blood pressure monitor 2. The blood pressure monitor 2 may measure blood pressure of the first subject from pulse transit time (PTT) or by using a method such as tonometry.

The mobile terminal 3 is, for example, a smart phone or a tablet, but is not limited thereto. The mobile terminal 3 receives blood pressure data from the blood pressure monitor 2 by using near-field wireless communication. The mobile terminal 3 associates the blood pressure data with identification information of the first subject who owns the mobile terminal 3 and transmits the blood pressure data to the server 1 over a network such as the Internet.

The information processing terminal 4 is, for example, a personal computer, but is not limited thereto. The information processing terminal 4 receives input by a physician. In one example, the physician makes a diagnosis that the subject has developed a disease and inputs, to the information processing terminal 4, information identifying the specific disease in association with identification information of the subject. The information processing terminal 4 transmits the information identifying the specific disease associated with the identification information of the subject to the server 1 over a network such as the Internet. As a result, the server 1 can store the first reference data for each subject in association with information indicating the onset of a specific disease.

### Server

### Hardware Configuration

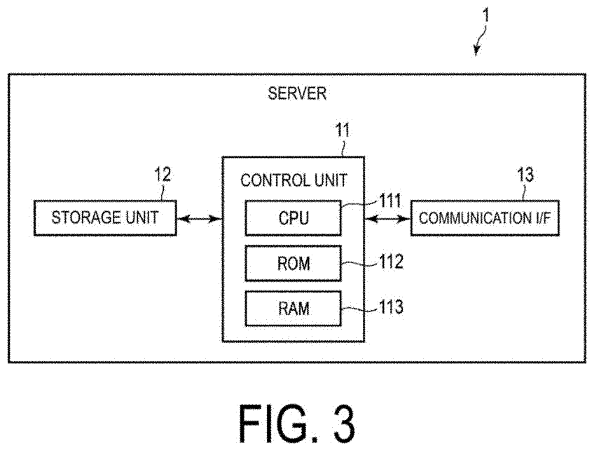

FIG. 3 is a diagram schematically illustrating an example of the hardware configuration of the server 1.

The server 1 is electrically connected to a control unit 11, a storage unit 12, and a communication interface 13. Note that in FIG. 3, the communication interface is described as a “communication I/F”.

The control unit 11 controls operation of each unit of the server 1. The control unit 11 includes a central processing unit (CPU) 111, a read only memory (ROM) 112 and a random access memory (RAM) 113. The CPU 111 is an example of a processor. The CPU 111 develops, to the RAM 113, a program that is stored in the storage unit 12 and causes the server 1 to function. Then, the CPU 111 interprets and executes the program developed to the RAM 113, and thus the control unit 11 can execute each unit described in the software configuration items.

The storage unit 12 is a so-called auxiliary storage device. The storage unit 12 is, for example, a hard disk drive (HDD), but is not limited thereto. The storage unit 12 stores the program to be executed by the control unit 11. This program causes the server 1 to function as each unit described in the software configuration items.

As exemplified below, the storage unit 12 stores various types of data used by the control unit 11.

The storage unit 12 stores the first blood pressure data related to the first subject. The control unit 11 stores blood pressure data in the storage unit 12 as part of the target data each time blood pressure data is received from the mobile terminal 3. Thus, the storage unit 12 stores the target data including a blood pressure value that has changed over time before the most recent blood pressure data is received.

The storage unit 12 stores one or more pieces of first reference data associated with information indicating the onset of a disease in each subject. Each piece of first reference data includes a blood pressure value that changes over time before a time at which each subject related to each piece of first reference data develops a disease.

The storage unit 12 stores a first management table. The first management table manages information related to each subject associated with each piece of first reference data. A configuration example of the first management table will be described later.

The communication interface 13 includes various wireless communication modules for a wireless local area network (WLAN) and mobile communication (3G, 4G, etc.). The communication interface 13 communicates with the mobile terminal 3 and the information processing terminal 4.

Note that components may be omitted, replaced or added to/from the specific hardware configuration of the server 1 as appropriate according to the embodiment. For example, the control unit 11 may include a plurality of processors.

### Configuration of First Management Table

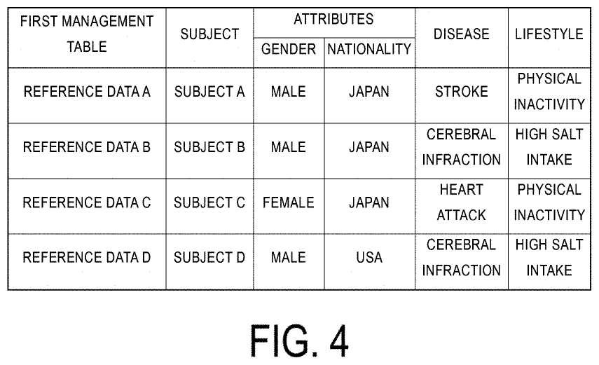

FIG. 4 is a diagram illustrating an example of the configuration of the first management table. The reference data A, reference data B, reference data C and reference data D illustrated in FIG. 4 each correspond to the first reference data. In the example of FIG. 4, the first management table includes information related to subjects each associated with one of four pieces of first reference data. The information related to the subject includes identification information of the subject, information indicating attributes of the subject, information indicating the disease that the subject has developed, and information indicating the lifestyle of the subject.

The identification information of the subject may be information indicating the name of the subject or information indicating an identification number of the subject. The identification information of the subject is based on information input by a physician or the subject.

The information indicating the attributes of the subject is information indicating characteristics of the subject. Attributes include gender and nationality. The attributes may include other elements in addition to at least one of the elements of gender and nationality, or in place of at least one of the elements of gender and nationality. The attributes may include the current age of the subject. The attributes may include the age at which the subject developed the disease. The information indicating the attributes of the subject is based on information input by a physician or the subject.

The information indicating the disease that the subject has developed is information identifying a specific disease. The information indicating the disease that the subject has developed is information indicating at least one of a stroke, a cerebral infarction, and a heart attack, but is not limited thereto. The information indicating the disease that the subject has developed is based on diagnostic results of a physician.

The information indicating the lifestyle of the subject is information indicating lifestyle before the subject developed the disease, which is assumed to have caused the onset of the disease in the subject. The information indicating the lifestyle of the subject is information indicating at least one of, for example, physical inactivity and high salt intake, but is not limited thereto. The information indicating the lifestyle of the subject is based on information input by a physician or the subject.

### Software Configuration

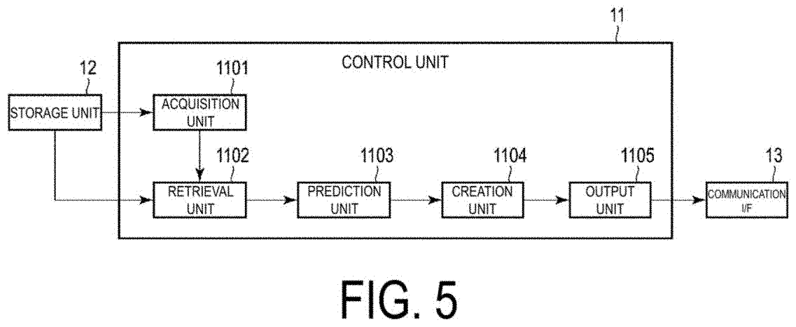

FIG. 5 is a diagram schematically illustrating an example of the software configuration of the server 1.

The control unit 11 implements an acquisition unit 1101, a retrieval unit 1102, a prediction unit 1103, a creation unit 1104, and an output unit 1105.

The acquisition unit 1101 will be described.

As exemplified below, the acquisition unit 1101 acquires the first blood pressure data related to the first subject. The acquisition unit 1101 acquires, from the storage unit 12, the first blood pressure data associated with the first subject. The acquisition unit 1101 outputs the first blood pressure data to the retrieval unit 1102.

The retrieval unit 1102 will be described.

The retrieval unit 1102 retrieves, from among the one or more pieces of first reference data stored in the storage unit 12, first reference data for which a degree of similarity to temporal change of the first blood pressure data satisfies a first condition. Hereinafter, the first reference data for which a degree of similarity to temporal change of the first blood pressure data satisfies a first condition will also be referred to as second blood pressure data. The subject related to the second blood pressure data will also be referred to as a second subject. The retrieval unit 1102 may retrieve second blood pressure data related to the second subject for which the degree of similarity to attributes of the first subject satisfies a second condition.

An example of the retrieval operation performed by the retrieval unit 1102 for the second blood pressure data will be described later.

The retrieval unit 1102 references the first management table based on retrieval of the second blood pressure data to acquire information related to the second subject associated with the second blood pressure data. The retrieval unit 1102 outputs the information related to the second subject to the prediction unit 1103.

The prediction unit 1103 will be described.

As exemplified below, the prediction unit 1103 predicts the onset of a particular disease in the first subject based on information indicating the onset of a particular disease in the second subject associated with the second blood pressure data. The prediction unit 1103 receives information related to the second subject from the retrieval unit 1102. The prediction unit 1103 acquires information indicating a disease that the second subject has developed from among the information related to the second subject. The prediction unit 1103 predicts that the first subject will develop the same disease as the particular disease that the second subject has developed. The prediction unit 1103 outputs, to the creation unit 1104, a prediction result that predicts onset of the particular disease in the first subject.

The creation unit 1104 will be described.

As exemplified below, the creation unit 1104 creates a first message including advice for the first subject based on the prediction result that predicts onset of the particular disease in the first subject. The creation unit 1104 receives the prediction result from the prediction unit 1103. The creation unit 1104 creates the first message including advice for the first subject based on the prediction result. The creation unit 1104 outputs the first message to the output unit 1105.

An example of the first message will be described.

The first message includes advice indicating that a subject is likely to develop a particular disease. The first message also includes advice on content related to prevention of the particular disease that the first subject is predicted to develop. In one example, the creation unit 1104 may create a first message including information based on the lifestyle of the second subject as advice. In this example, the creation unit 1104 creates the first message by referencing the information indicating the lifestyle of the second subject stored in the storage unit 12. In another example, the creation unit 1104 may create a first message including general information for preventing a particular disease as advice. In this example, the creation unit 1104 creates the first message by referencing general information for preventing the particular disease stored in the storage unit 12.

The output unit 1105 will be described.

As exemplified below, the output unit 1105 outputs the first message. The output unit 1105 receives the first message from the creation unit 1104. The output unit 1105 outputs the first message to the communication interface 13. The communication interface 13 transmits the first message to the mobile terminal 3 over a network. With this configuration, the first subject can see the first message by using the mobile terminal 3.

### § 3 OPERATION EXAMPLE

### Server—Retrieval Operation for Second Blood Pressure Data

An example of the retrieval operation for the second blood pressure data performed by the retrieval unit 1102 will be described.

First, the retrieval unit 1102 receives the first blood pressure data from the acquisition unit 1101.

Then, the retrieval unit 1102 compares temporal change of the first blood pressure data to temporal change of each piece of first reference data stored in the storage unit 12. The retrieval unit 1102 uses the temporal change of the first blood pressure data within a comparison period retroactive from the present time as a comparison target. The comparison period is, for example, six months, one year or five years, but is not limited thereto. The length of the comparison period can be arbitrarily set. The retrieval unit 1102 compares the first blood pressure data to each piece of the first reference data on change in at least one of the systolic blood pressure SBP and the diastolic blood pressure DBP.

Next, the retrieval unit 1102 evaluates the degree of similarity between the temporal change of the first blood pressure data and the temporal change of each piece of first reference data. In one example, the retrieval unit 1102 evaluates the degree of similarity in consideration of at least similarity between trends of change. In another example, the retrieval unit 1102 evaluates the degree of similarity in consideration of similarity between values in addition to similarity between the trends of change. The retrieval unit 1102 can use known technology for evaluating the degree of similarity between waveforms.

Next, the retrieval unit 1102 determines whether the degree of similarity between the first blood pressure data and each piece of the first reference data satisfies a first condition. Then, the retrieval unit 1102 retrieves, as second blood pressure data, first reference data for which the degree of similarity to temporal change of the first blood pressure data satisfies the first condition.

The first condition will be described.

The first condition includes a definition related to a degree of similarity threshold. The same threshold or different thresholds may be used for the systolic blood pressure SBP and the diastolic blood pressure DBP. The threshold may vary depending on the length of the comparison period. For example, the threshold may be set to be lower as the comparison period increases. This is because each piece of the first reference data is less likely to be similar to the first blood pressure data as the comparison period increases.

The first condition may include a definition specifying a target for which the degree of similarity is to be evaluated. The target for which the degree of similarity is to be evaluated is at least one of the systolic blood pressure SBP and the diastolic blood pressure DBP. The first condition may include a definition other than a definition that specifies a target for which the degree of similarity is to be determined.

As described above, the retrieval unit 1102 can retrieve the second blood pressure data that satisfies the first condition among the one or more pieces of first reference data stored in the storage unit 12.

Note that the retrieval unit 1102 may retrieve second blood pressure data related to the second subject for which the degree of similarity to the attributes of the first subject satisfies the second condition.

The second condition will be described.

The second condition includes a definition related to determination criteria for degree of similarity. Some examples of the determination criteria for degree of similarity are given below, but the determination criteria is not limited to these examples. The determination criteria for degree of similarity may be a number of matched elements between two subjects. The determination criteria for degree of similarity may be a ratio of elements that match between two subjects among a plurality of previously specified elements. The determination criteria for degree of similarity may be a perfect match between two subjects for one or more specified elements.

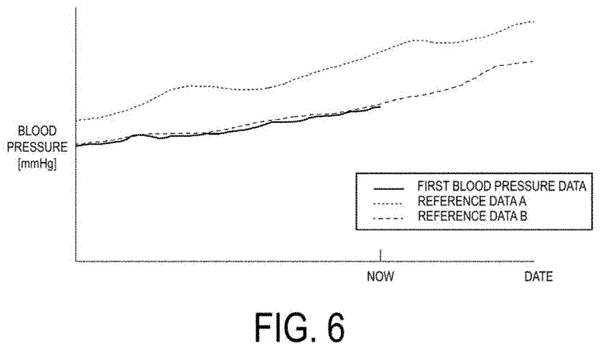

Next, an example of retrieval of the second blood pressure data by the retrieval unit 1102 in consideration of the attributes of the first subject will be described with reference to FIG. 6.

FIG. 6 is a graph showing an example of comparison of the first blood pressure data to the reference data A and the reference data B. FIG. 6 shows temporal change of the systolic blood pressure SBP. Note that temporal change of the diastolic blood pressure DBP is omitted for the sake of simplicity.

It is assumed that the gender of the first subject is male and the nationality of the first subject is Japanese. The second condition is assumed to include a definition related to complete match for gender and nationality.

First, the retrieval unit 1102 receives the first blood pressure data from the acquisition unit 1101.

Then, the retrieval unit 1102 extracts, from among the reference data A, the reference data B, the reference data C, and the reference data D stored in the storage unit 12, the reference data A and the reference data B related to respective subjects for which the degree of similarity to attributes of the first subject satisfies the second condition.

As illustrated in FIG. 6, the retrieval unit 1102 compares the temporal change of the first blood pressure data to the temporal change of each piece of the reference data A and the reference data B.

Then, the retrieval unit 1102 evaluates the degree of similarity between the temporal change of the first blood pressure data and the temporal change of each piece of the reference data A and the reference data B.

Next, the retrieval unit 1102 determines whether the degree of similarity between the first blood pressure data and each piece of the reference data A and the reference data B satisfies the first condition. Here, it is assumed that the degree of similarity between the first blood pressure data and the reference data A does not satisfy the first condition. It is assumed that the degree of similarity between the first blood pressure data and the reference data B satisfies the first condition. The retrieval unit 1102 retrieves, as the second blood pressure data, the reference data B for which the degree of similarity to the temporal change of the first blood pressure data satisfies the first condition.

### Prediction Operation for Onset of Disease

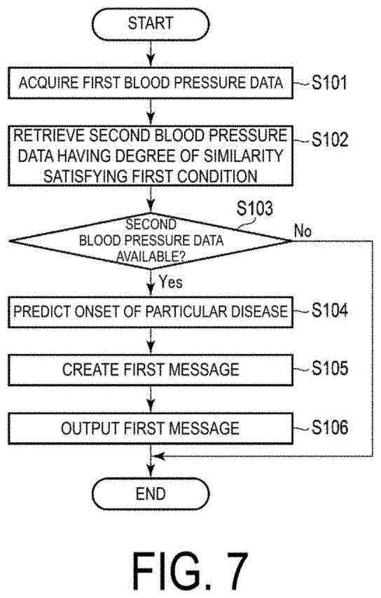

FIG. 7 is a flowchart illustrating an example of a prediction operation, by the server 1, for onset of a disease. Note that the processing procedure described below is merely exemplary, and each of the processes may be changed to the greatest extent possible. Further, steps may be omitted, substituted or added as appropriate to/from the processing procedure described below.

As described above, the acquisition unit 1101 acquires the first blood pressure data related to the first subject (Step S101).

As described above, from among one or more pieces of first reference data related to one or more subjects different from the first subject and each being associated with information indicating the onset of a disease in each subject, the retrieval unit 1102 retrieves second blood pressure data related to the second subject and for which the degree of similarity to temporal change of the first blood pressure data satisfies the first condition (Step S102). In Step S102, for example, the retrieval unit 1102 retrieves reference data as the second blood pressure data that satisfies the first condition from among the reference data A, the reference data B, the reference data C, and the reference data D.

The retrieval unit 1102 determines whether it was possible to retrieve the second blood pressure data as described above (Step S103). In Step S103, for example, the retrieval unit 1102 retrieves the reference data B as the second blood pressure data. When the retrieval unit 1102 is unable to retrieve the second blood pressure data (Step S103, No), the retrieval unit 1102 ends the retrieval operation for second blood pressure data.

When the retrieval unit 1102 is able to retrieve the second blood pressure data (Step S103, Yes), the prediction unit 1103 predicts the onset of a particular disease in the first subject based on information indicating the onset of a particular disease in the second subject associated with the second blood pressure data (Step S104). In Step S104, for example, the prediction unit 1103 predicts the onset of a cerebral infarction in the first subject.

As described above, the creation unit 1104 creates the first message including advice for the first subject based on the prediction result that predicts the onset of a particular disease in the first subject (Step S105). In Step S105, for example, the creation unit 1104 creates a first message including advice indicating that the subject is likely to develop a cerebral infarction. For example, the creation unit 1104 creates a first message including advice indicating reduction in salt intake in order to prevent the onset of a cerebral infarction.

The output unit 1105 outputs the first message as described above (Step S106).

In Step S106, the output unit 1105 may output the first message based on an instruction indicating permission of a physician to output the first message. In this case, the server 1 transmits, to the information processing terminal 4, a message asking for permission to output the first message. The physician inputs the instruction indicating permission to output the first message to the information processing terminal 4. The information processing terminal 4 outputs the instruction indicating permission to output the first message to the server 1. The server 1 outputs the first message based on reception of the instruction indicating permission to output the first message. This operation prevents the first message from being displayed to the first subject without the permission of a physician. Note that permission to output the first message is not limited to permission of a physician. Depending on the content of the first message, the permission to output the first message may be permission of medical personnel, such as a nurse or a public health nurse.

Note that the server 1 may perform a plurality of the prediction operations for onset of a disease exemplified in FIG. 7 for the first subject. Thus, the server 1 may output the first message again according to the prediction operation of onset of a disease exemplified in FIG. 7 after the first message is output for a first time.

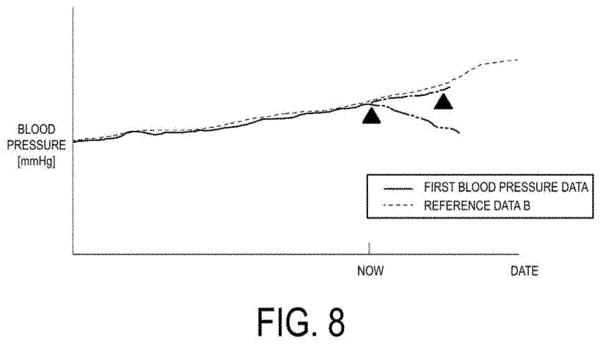

FIG. 8 is a graph showing an example of output timing of the first message. The black triangle in FIG. 8 indicates the output timing of the first message. FIG. 8 shows the temporal change of the systolic blood pressure SBP. Note that temporal change of the diastolic blood pressure DBP is omitted for the sake of simplicity.

First, after the server 1 outputs the first message for a first time, it is assumed that the first blood pressure data follows the change indicated by the dot-dash line. It is also assumed that the degree of similarity between the change indicated by the dot-dash line and the temporal change of the reference data B satisfies the first condition. After outputting the first message for a first time, the server 1 performs the prediction operation for onset of a disease exemplified in FIG. 7 at arbitrary timing. The server 1 still predicts the onset of a cerebral infarction in the first subject and outputs the first message again.

Next, after the server 1 outputs the first message for a first time, it is assumed that the first blood pressure data follows the change indicated by the two-dot chain line. It is also assumed that the degree of similarity between the change indicated by the two-dot chain line and the temporal change of the reference data B does not satisfy the first condition. After outputting the first message for a first time, the server 1 performs the prediction operation for onset of a disease exemplified in FIG. 7 at arbitrary timing. The server 1 does not predict the onset of a cerebral infarction in the first subject, and thus does not output the first message again.

### Actions and Effects

As described above, in the present embodiment, the server 1 retrieves the second blood pressure data that is related to the second subject and satisfies the first condition in terms of degree of similarity to temporal change of the first blood pressure data related to the first subject, to thereby predict the onset of a particular disease in the first subject.

With this configuration, by retrieving the second blood pressure data for which the degree of similarity to the temporal change of the first blood pressure data satisfies the first condition, the sever 1 can more accurately predict the onset of a particular disease in the first subject.

In addition, in the present embodiment, the server 1 retrieves the second blood pressure data that is related to the second subject and has a degree of similarity to the attributes of the first subject that satisfies the second condition.

With this configuration, by retrieving the second blood pressure data in consideration of the attributes of the first subject, the sever 1 can more accurately predict the onset of a particular disease in the first subject. This is because, even when there are a plurality of pieces of blood pressure data indicating the onset of a particular disease, the temporal change of each piece of blood pressure data varies depending on the attributes of the subject related to each piece of blood pressure data.

In the present embodiment, the server 1 creates the first message including advice for the first subject based on the prediction result that predicts the onset of a particular disease in the first subject.

With this configuration, the server 1 can provide the first subject with the first message before the first subject develops the particular disease. As a result, the first subject can prevent the onset of a particular disease by referring to the advice included in the first message.

Further, in the present embodiment, the first message is created including information based on the lifestyle of the second subject as advice.

With this configuration, the first subject can refer to the information based on the lifestyle of the second subject who has developed a particular disease to reconsider their lifestyle and prevent the onset of the particular disease.

Further, in the present embodiment, the first message is output based on an instruction indicating permission of medical personnel to output the first message.

With this configuration, the server 1 can present the first message to the first subject in compliance with laws and regulations even when permission of medical personnel is required by laws and regulations to present the first message to the first subject. As a result, the first subject can obtain a high quality first message with the permission of medical personnel.

### § 4 MODIFIED EXAMPLES

### 4-1 Modified Example 1

As exemplified below, the server 1 is configured to determine whether the first subject is still likely to develop a particular disease after the first message is output.

### § 4-1-1 Configuration Example—Server

### Hardware Configuration

The server 1 includes the units that are exemplified in FIG. 3 and described in the above-described embodiment.

The storage unit 12 stores the various types of data described below in addition to the various types of data described in the above-described embodiment.

The storage unit 12 stores information that identifies the first reference data retrieved as the second blood pressure data in association with the first blood pressure data. For example, the first blood pressure data is associated with information that identifies the reference data B retrieved as the second blood pressure data.

The storage unit 12 stores one or more pieces of second reference data in addition to the one or more pieces of first reference data managed in the first management table. The one or more pieces of second reference data differ from the one or more pieces of first reference data in that each piece of second reference data is not associated with information indicating the onset of a disease in each subject. The second reference data includes a blood pressure value that changes over time. The second reference data is associated with at least one instance of output of the first message.

The storage unit 12 stores a second management table in addition to the first management table. The second management table manages information related to the subjects associated with each piece of second reference data. A configuration example of the second management table will be described later.

### Configuration of Second Management Table

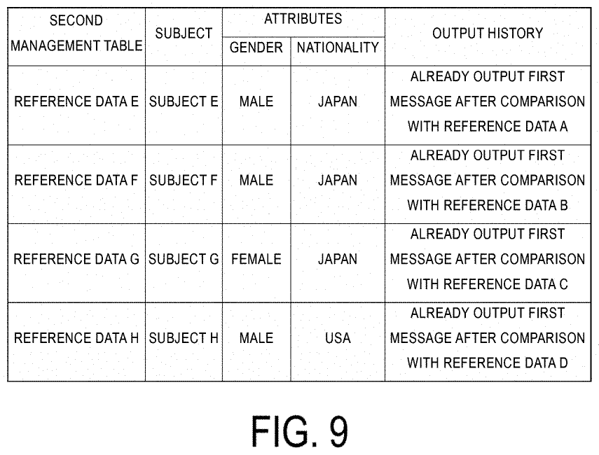

FIG. 9 is a diagram illustrating an example of the configuration of the second management table. The reference data E, reference data F, reference data G and reference data H illustrated in FIG. 9 each correspond to the second reference data. In the example of FIG. 9, the second management table includes information related to subjects each associated with one of four pieces of second reference data. The information related to the subject includes identification information of the subject, information indicating attributes of the subject, and information indicating output history.

The identification information of the subject and the information indicating attributes of the subject are as described above in the present embodiment.

The information indicating output history includes information indicating that the first message has been output. The information indicating output history also includes information identifying first reference data retrieved as the second blood pressure data for output of the first message. For example, the reference data E is associated with information indicating that the first message has been output at least once. The reference data E is also associated with information identifying the reference data A retrieved as the second blood pressure data for output of the first message.

### Software Configuration

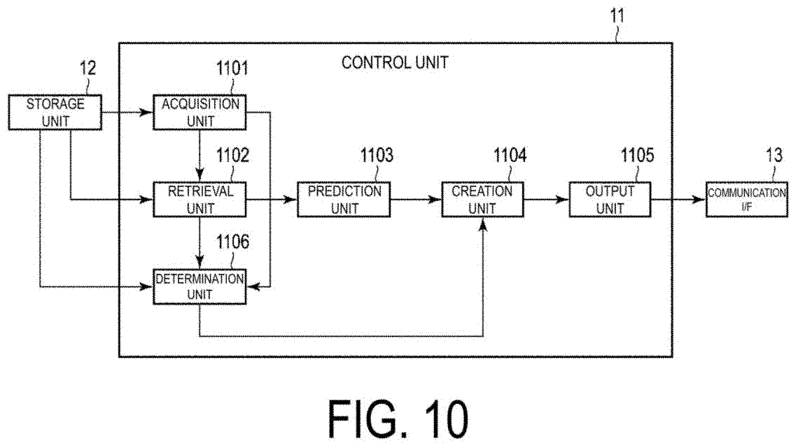

FIG. 10 is a diagram schematically illustrating an example of the software configuration of the server 1.

The control unit 11 implements a determination unit 1106 in addition to the acquisition unit 1101, the retrieval unit 1102, the prediction unit 1103, the creation unit 1104, and the output unit 1105 described above in the present embodiment.

The determination unit 1106 will be described.

The determination unit 1106 determines whether the first blood pressure data is similar to the second blood pressure data or third blood pressure data after the first message is output. Here, the third blood pressure data is second reference data associated with first reference data retrieved as the second blood pressure data from among the one or more pieces of second reference data managed in the second management table. The third blood pressure data relates to a subject different from the first subject related to the first blood pressure data and the second subject related to the second blood pressure data.

An example of the determination operation performed by the determination unit 1106 will be described later.

The determination unit 1106 outputs the determination result to the creation unit 1104. The determination result indicates whether the temporal change of the first blood pressure data after the first message is output is similar to either the second blood pressure data or the third blood pressure data.

The creation unit 1104 will be described.

As exemplified below, in addition to creating the first message, the creation unit 1104 creates a second message including different advice for the first subject according to the determination result. The creation unit 1104 receives the determination result from the determination unit 1106. The creation unit 1104 creates the second message including different advice according to the determination result. The creation unit 1104 outputs the second message to the output unit 1105.

An example of the second message will now be described.

First, a description will be given of a case where the determination result indicates that the temporal change of the first blood pressure data after the first message is output is similar to the second blood pressure data. The second message includes advice indicating that the subject is still likely to develop a particular disease. Similar to the first message, the second message also includes advice on content related to prevention of a particular disease that the first subject is predicted to develop.

Next, a description will be given of a case where the determination result indicates that the temporal change of the first blood pressure data after the first message is output is similar to the third blood pressure data. The second message includes advice indicating that the subject is less likely to develop a particular disease.

The output unit 1105 will be described.

As exemplified below, the output unit 1105 outputs the second message in addition to the first message. The output unit 1105 receives the second message from the creation unit 1104. The output unit 1105 outputs the second message to the communication interface 13. The communication interface 13 transmits the second message to the mobile terminal 3 over a network. With this configuration, the first subject can see the second message by using the mobile terminal 3.

### § 4-1-2 Operation Example

### Server

### Determination Operation

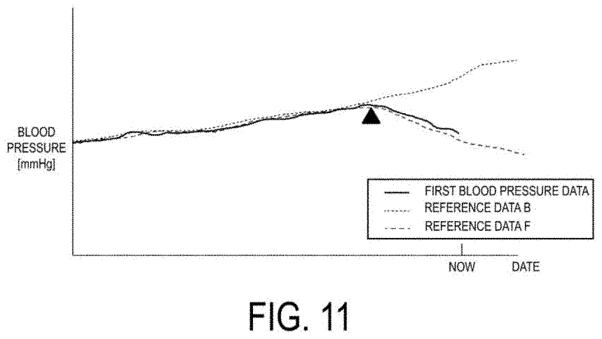

The determination operation performed by the determination unit 1106 will be described with reference to FIG. 11.

FIG. 11 is a graph showing an example of comparison of the first blood pressure data to the reference data B and the reference data F. FIG. 11 shows the temporal change of the systolic blood pressure SBP. Note that temporal change of the diastolic blood pressure DBP is omitted for the sake of simplicity.

The determination unit 1106 performs a determination operation for the first blood pressure data at a predetermined timing after the first message is output. The predetermined timing is, for example, an end point of an elapsed period after the first message is output, such as one month, six months or one year. The predetermined timing can be arbitrarily set.

First, the determination unit 1106 receives the first blood pressure data from the acquisition unit 1101.

Then, the determination unit 1106 acquires, from the storage unit 12, the first reference data retrieved as the second blood pressure data for output of the first message. For example, the determination unit 1106 references information identifying the reference data B associated with the first blood pressure data stored in the storage unit 12. The determination unit 1106 identifies that the first reference data retrieved as the second blood pressure data for output of the first message is the reference data B. The determination unit 1106 acquires the reference data B from the storage unit 12.

Next, the determination unit 1106 acquires the second reference data as the third blood pressure data from the storage unit 12. For example, the determination unit 1106 references the information indicating the output history of the second management table and acquires the reference data F associated with the reference data B, which is the second blood pressure data, from the storage unit 12 as the third blood pressure data.

Next, as illustrated in FIG. 11, the determination unit 1106 determines whether the first blood pressure data after the first message is output is similar to either the second blood pressure data or the third blood pressure data. For example, the determination unit 1106 determines whether the first blood pressure data after the first message is output is similar to either the reference data B or the reference data F. The determination unit 1106 can use known technology for evaluating the degree of similarity between waveforms.

### Prediction Operation for Onset of Disease

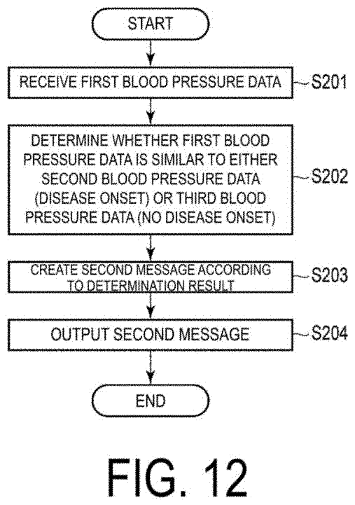

FIG. 12 is a flowchart illustrating an example of the prediction operation for onset of a disease performed by the server 1. Note that the processing procedure described below is merely exemplary, and each of the processes may be changed to the greatest extent possible. Further, steps may be omitted, substituted or added as appropriate to/from the processing procedure described below.

As described above, the determination unit 1106 receives the first blood pressure data after the first message is output (Step S201).

As described above, the determination unit 1106 determines whether the first blood pressure data after the first message is output is similar to either the second blood pressure data or the third blood pressure data (Step S202).

As described above, the creation unit 1104 creates a second message including advice indicating different content for the first subject according to the determination result indicating whether the temporal change of the first blood pressure data after the first message is output is similar to either the second blood pressure data or the third blood pressure data (Step S203).

The output unit 1105 outputs the second message as described above (Step S204).

In Step S204, the output unit 1105 may output the second message based on an instruction indicating permission of a physician to output the second message, similar to output of the first message.

### Actions and Effects

As described above, in Modified Example 1, the server 1 determines whether the first blood pressure data after the first message is output is similar to either the second blood pressure data associated with information indicating the onset of a disease or the third blood pressure data not associated with information indicating the onset of a disease.

With this configuration, the server 1 can determine whether the first subject is still likely to develop a particular disease after the first message is output. As a result, the first subject can refer to the second message output after the first message to determine whether they are still likely to develop a particular disease.

### 4-2 Modified Example 2

Blood pressure data has been described as an example in the present embodiment, but the present invention is not limited thereto. The present embodiment can also be applied to biometric data other than blood pressure data. Biometric data may be data related to an electrocardiogram or pulse rate. Thus, the term “blood pressure data” in the present embodiment may be regarded as “biometric data”.

### 4-3 Modified Example 3

In summary, the present invention is not limited to the above-described embodiment and components can be modified and embodied within a scope that does not depart from the gist of the present invention. Further, various inventions can be formed by appropriately combining a plurality of the components disclosed in the above-described embodiment. For example, some components of the total number of components illustrated in the embodiments may be deleted. Furthermore, components of different embodiments may be combined as appropriate.

### § 5 NOTES

The present embodiment can be partly or entirely described as indicated in both the claims and the following Note, but is not limited to such description.

### Note

A data processing device (1) including: an acquisition unit (1101) configured to acquire first biometric data related to a first subject, a retrieval unit (1102) configured to retrieve, from among one or more pieces of biometric data, second biometric data that is related to a second subject and has a degree of similarity to temporal change of the first biometric data that satisfies a first condition, the one or more pieces of biometric data related to one or more subjects different from the first subject and associated with information indicating onset of a disease in each of the one or more subjects, and a prediction unit (1103) configured to predict onset of a particular disease in the first subject based on information indicating onset of the particular disease in the second subject associated with the second biometric data.

### REFERENCE SIGNS LIST

1 Server
2 Blood pressure monitor
3 Mobile terminal
4 Information processing terminal
11 Control unit
12 Storage unit
13 Communication I/F
111 CPU
112 ROM
113 RAM
1101 Acquisition unit
1102 Retrieval unit
1103 Prediction unit
1104 Creation unit
1105 Output unit
1106 Determination unit
S Server
S1 Acquisition unit
S2 Retrieval unit
S3 Prediction unit

1. A data processing device comprising:
an acquisition unit configured to acquire first biometric data related to a first subject;
a retrieval unit configured to retrieve, from among one or more pieces of biometric data, second biometric data that is related to a second subject and has a degree of similarity to temporal change of the first biometric data that satisfies a first condition, the one or more pieces of biometric data related to one or more subjects different from the first subject and associated with information indicating onset of a disease in each of the one or more subjects;
a prediction unit configured to predict onset of a particular disease in the first subject based on information indicating onset of the particular disease in the second subject associated with the second biometric data;
a creation unit configured to create a first message including advice for the first subject based on a prediction result that predicts onset of the particular disease in the first subject;
an output unit configured to output the first message; and
a determination unit configured to determine whether the first biometric data after the first message is output is similar to either the second biometric data or third biometric data that is related to a third subject and not associated with information indicating the onset of a disease, wherein
the creation unit is configured to create a second message including advice indicating different content for the first subject according to a determination result indicating whether temporal change of the first biometric data after the first message is output is similar to either the second biometric data or the third biometric data, and
the output unit is configured to output the second message.

2. The data processing device according to claim 1, wherein the retrieval unit is configured to retrieve the second biometric data that is related to the second subject and has a degree of similarity to an attribute of the first subject that satisfies a second condition.

3. The data processing device according to claim 1, wherein the creation unit is configured to create the first message including information based on lifestyle of the second subject as the advice.

4. The data processing device according to claim 1, wherein the output unit is configured to output the first message based on an instruction indicating permission of medical personnel to output the first message.

5. A data processing method comprising:
acquiring first biometric data related to a first subject;
retrieving, from among one or more pieces of biometric data, second biometric data that is related to a second subject and has a degree of similarity to temporal change of the first biometric data that satisfies a first condition, the one or more pieces of biometric data related to one or more subjects different from the first subject and associated with information indicating onset of a disease in each of the one or more subjects;
predicting onset of a particular disease in the first subject based on information indicating onset of the particular disease in the second subject associated with the second biometric data;
creating a first message including advice for the first subject based on a result of the prediction that predicts onset of the particular disease in the first subject;
outputting the first message; and
determining whether the first biometric data after the first message is output is similar to either the second biometric data or third biometric data that is related to a third subject and not associated with information indicating the onset of a disease, wherein
in the creating, a second message including advice indicating different content for the first subject is created according to a determination result indicating whether temporal change of the first biometric data after the first message is output is similar to either the second biometric data or the third biometric data, and
in the outputting, the second message is output.

6. A non-transient storage medium storing a data processing program that causes a computer to function as each unit included in the data processing device of claim 1.

7. The data processing device according to claim 1, wherein, when it is determined that temporal change of the first biological data after the first message is output is similar to the third biometric data, the creation unit is configured to create the second message including advice indicating that onset of the particular disease is less likely.

8. A non-transient storage medium storing a data processing program that causes a computer to function as each unit included in the data processing device of claim 2.

9. A non-transient storage medium storing a data processing program that causes a computer to function as each unit included in the data processing device of claim 3.

10. A non-transient storage medium storing a data processing program that causes a computer to function as each unit included in the data processing device of claim 4.
